(19) United States
(12) Patent Application Publication
(10) Pub. No.: US 2011/0059462 A1
(43) Pub. Date: Mar. 10, 2011

(54) AUTOMATED PARTICULATE CONCENTRATION SYSTEM

(75) Inventors: Daniel V. Lim, Tampa, FL (US); Elizabeth A. Kearns, Tampa, FL (US); Stephaney D. Leskinen, Wesley Chapel, FL (US); Sonia Magaña, Tampa, FL (US)

(73) Assignee: UNIVERSITY OF SOUTH FLORIDA, Tampa, FL (US)

(21) Appl. No.: 12/945,444

(22) Filed: Nov. 12, 2010

Related U.S. Application Data
(63) Continuation-in-part of application No. 11/841,215, filed on Aug. 20, 2007.
Application No. 11/841,215 is a continuation of application No. PCT/US2006/006002, filed on Feb. 21, 2006.

(51) Int. Cl. C12Q 1/68; B01D 35/00; G01N 33/00; C12M 1/34; G01N 21/76; G01N 1/00; C12Q 1/02; G01N 1/30; G01N 33/53; G01N 21/00; G01N 21/62; G01N 24/00

(52) U.S. Cl. 435/6; 422/534; 422/68.1; 435/287.1; 422/52; 435/287.2; 436/178; 435/29; 435/40.5; 436/94; 436/127; 435/7.92; 436/172; 436/164; 436/171; 436/173

(57) ABSTRACT

An automated system for concentrating potentially harmful substances from various water types or other non-viscous liquids to facilitate detection of those substances is disclosed herein. The automated system comprises a water pressure driven or pump driven concentration unit that filters the test fluid through a hollow-fiber filter. Material collected on the filter is backflushed into a collection vessel by passing a small volume of sterile solution through the filter in the reverse direction. The automated system can be configured to be portable or to be integrated into a continuous liquid stream for online monitoring of test fluids. Optionally, an electronic signal at the end of the backflush sequence triggers a detector, such as an automated array biosensor, to begin processing and analyzing the sample.

### CROSS REFERENCE TO RELATED APPLICATION

This application is a continuation-in-part of U.S. application Ser. No. 11/841,215, entitled “Automated Concentration System”, filed Aug. 20, 2007, which is a continuation of International Patent Application PCT/US2006/006002, entitled “Automated Concentration System”, filed Feb. 21, 2006 which claims priority to U.S. Provisional Patent Application 60/593,484, entitled “Automated Concentration System”, filed Feb. 18, 2005; which is fully incorporated herein by reference.

### GOVERNMENT SUPPORT

This invention was developed under support from: the U.S. Army Research, Development and Engineering Command (RDECOM) under grant DAAD13-00-C-0037, accordingly the U.S. government may have certain rights in the invention; and Pinellas County Utilities under grant 1209-101-700, who may have certain rights in the invention.

### FIELD OF INVENTION

This invention relates to particulate and biologic concentration systems. More specifically, the invention provides a novel concentration system designed to collect and concentrate particulates and organisms from a liquid for testing or analysis.

### BACKGROUND OF THE INVENTION

The safety of drinking water has long been a concern of water utilities and other government entities. Current analysis methods take several days to accomplish and there is a desire for more rapid methods of determining when a potential health hazard is present in a water supply. In addition, portable water supplies are considered part of the U.S. critical infrastructure that has been mandated to increase security since Sep. 11, 2001. Military services are also concerned about the security of this critical resource at military bases and temporary field military installations.

The prior art describes methods using hollow-fiber filter ultra-filtration to concentrate microorganisms from water for subsequent detection. Previous methods, however, require manual control of the system; none are amenable to being automated. Previous attempts to detect the presence of microorganisms require the sample to be transported to a remote location to be tested. Existing systems also require pretreatment of the filter prior to concentration in order to achieve adequate concentration of the targeted microorganisms. Pre-treatment increases the complexity of the concentration process and prevents automation of the system.

Therefore, what is needed is an automated device that is capable of being placed online in a flow system to monitor for the presence of microorganisms.

### SUMMARY OF THE INVENTION

This invention provides a method of concentrating particulates, including hazardous biological material like bacteria, viruses and toxins, from liquid sources, like water supplies. The inventive system includes a water concentration system to facilitate the detection of potentially harmful substances. The system optionally uses a pressure-driven unit that filters water or other fluid through a hollow-fiber filter. Alternatively, a pump may drive test fluid, such as water or other fluid, into the system from a water source. Material collected within the filter is backflushed into a collection vessel by passing a sterile solution through the filter in the reverse direction. The concentrator comprises a filter with a test fluid input port disposed on one side and test fluid output port disposed on the other side of the filter. A series of backflush subsystems operate in conjunction with the concentrator, allowing liquid backflush and air backflush regimens to flush the particulates from the filter, which are collected as a concentrated retentate in a sample output. The system is optionally coupled to a detector that screens the retentate for the presence of designated hazardous substances. An electronic signal can be delivered at the end of the backflush sequence to trigger a detector, such as a biosensor, to begin analyzing the sample.

The backflush subsystems may be operated using at least one pump in fluid communication with at least the liquid backflush or air backflush subsystem. Exemplary pumps include metering pumps, bellow pumps, double-diaphragm pumps, flexible impeller pumps, rotary lobe pumps, rotary vane pumps, oscillating pumps, piston pumps, syringe pumps, nutating disc pumps, flexible liner pumps, progressing cavity pumps, and peristaltic pumps. In certain variations of the air backflush subsystems, ambient air is collected and used as the air backflush, with an air intake valve used to collect ambient air during an air backflush. The system may include an air filter on the intake valve to limit contamination of the concentrator or retentate sample. The air backflush system may alternatively use other air sources, such as a pressurized gas container. The liquid backflush subsystem may use a pump drawing a recovery fluid from a liquid solution reservoir.

Liquid is directed through the concentrator using valves. Exemplary valves include butterfly valves, trunnions, ball valves, plug valves, globe valves, solenoid valves, needle valves, check valves, gate valves, angle seat piston valves, angle valves, ceramic disc valves, piston valves, and pinch valves adapted to control the flow of the test fluid through the apparatus, and mass flow controllers.

The concentrator may also utilize at least one pressure monitor to determine the status of the system. It has been found that placing one pressure monitor before the filter and one after the filter, along the path of the test fluid, allows the system to determine flow of liquid through the filter and calculate when backflushes and/or cleaning cycles are required. Exemplary pressure monitors include transducers, piezo-resistive pressure sensors, piezo-resistive pressure transducers, miniature cylindrical pressure transducers, silicon strain gauge pressure transducers, pressure transmitters, digital pressure gauges, and analog pressure gauges.

Users can continuously concentrate potentially hazardous materials from a water source for a desired amount of time by placing it in the water flow path or by diverting a subset of the water flow to the concentrator. For example, the device could be placed in the public drinking water distribution system and used to monitor the security of this critical resource. While the protection of portable water resources provides the broadest benefit, other types of water or liquid streams can also be monitored using this technology and multiple uses are contemplated.

The concentrator includes an output to allow recovery of the retentate containing the collected analyte. Any known sample detection system may be used to analyze the retentate for the analyte of interest, such as an array biosensor housing a slide prepared with antibodies to the test organism. The biosensor may be programmed to automatically run sample and detection reagents over the slide, analyze the resulting pattern for positive and negative data, and report the results. Other examples of sample detection systems include automated sensors, ELISA, chemiluminescent methods, differential staining, or nucleic acid amplification.

The inventive system removes any material, including hazardous material, suspended in the fluid that is greater than the pore size of the filter. The use of subsystems makes filter pretreatment unnecessary. However, the concentrator may include a large-particulate prefilter disposed along the path of the test fluid and before the test fluid reaches the filter, which may be used to remove coarse contaminants from the liquid input. The need for any water pre-filtration depends on numerous factors, such as the type and quality of the water to be tested. Further, the selection of a prefilter is determined by the factors influencing water quality and that might adversely impact filtration. Exemplary filters include size-exclusion filters and ion exchange filters.

The concentrator may also include a forward flow buffer subsystem. The forward flow buffer subsystem comprises a solution reservoir connected to a pump, and in fluid communication with the test fluid input line. In addition to, or in place of, the forward flow buffer subsystem, the concentrator may also include a forward flow air subsystem, which uses an air source in fluid communication with the test fluid input line and before the test fluid input port. While any known gas source may be used, exemplary gas sources are a pump in fluid communication with an air-intake valve, and a pressurized gas container. In systems using one or both of the forward flow systems, the systems are integrated into the concentrator so that valves shut off the test fluid before initiating the forward flow subsystem. This permits the system to equilibrate the filter to the recovery fluid and/or remove test fluid or recovery fluid from the filter prior to backflushing.

The system permits a user to extract an analyte from a test fluid by providing a test fluid source to the concentrator. The test fluid is passed along a first path of travel through a filter, capturing an analyte on the first side of the filter. The filter containing an analyte is equilibrated to a recovery fluid by flowing the recovery fluid over the filter in the same direction as the test fluid. The recovery fluid is removed by flowing air in the same direction as the test fluid and at least one backflush sequence is used to remove the retentate sample containing the analyte from the filter. The backflush sequence comprises at least one air backflush and a plurality of liquid backflushes. Air backflush may utilize ambient air; and liquid backflushes may use water, a buffer or other solution. The backflushed material is then collected as a concentrated sample (the retentate). In exemplary embodiments, the retentate is analyzed by culturing, automated sensors, ELISA, chemiluminescent methods, differential staining, or nucleic acid amplification. However, other known methods of detecting contaminants in liquids are envisioned.

Analysis of the retentate thereby alerts a user to any hazardous material discovered and identified. The process is automated and requires an attendant when a harmful material is discovered or if maintenance is required.

During initiation of test fluid flow, the user may purge any gas accumulated in the filter. Additionally, the system may undergo a cleaning prior to reinitiation of the test fluid flow. In these situations, a cleaning solution is pumped through the filter, optionally in the same path of travel as the test fluid. Gas may need to be purged from the filter at the initiation of cleaning solution flow. The cleaning solution may also be heated before cleaning.

### BRIEF DESCRIPTION OF THE DRAWINGS

For a fuller understanding of the invention, reference should be made to the following detailed description, taken in connection with the accompanying drawings, in which:

FIG. 1 is a schematic representation of an embodiment of the invention showing the integrated system.

FIG. 2 is a schematic representation of the invention showing the flow path of the forward flow concentration subsystem.

FIG. 3A is a schematic representation of the invention showing the flow path of the air backflush subsystem.

FIG. 3B is a schematic representation of the invention showing the flow path of the liquid backflush subsystem.

FIG. 4 is a schematic representation of the invention showing the flow path of the cleaning subsystem.

FIG. 5 is a schematic representation of the invention showing the flow path of the purge subsystem.

FIG. 6 is a schematic representation of an embodiment of the invention showing the integrated system.

FIG. 7 is a schematic representation of the invention showing the flow path of liquid through the forward flow recovery subsystem.

FIG. 8 is a schematic representation of the invention showing the flow path of air through the forward flow recovery subsystem.

FIG. 9A is a graph showing data from use of the inventive method to concentrate indicator organisms from river water at a low impact site.

FIG. 9B is a graph showing data from use of the inventive method to concentrate indicator organisms from river water at a high impact site.

### DETAILED DESCRIPTION OF THE PREFERRED EMBODIMENT

As used herein, “dead-end ultrafiltration” is a process of filtering fluid through an ultrafilter in a single path without recirculation of the feed (test) fluid and removing particulates from that ultrafilter via backflushing.

The term “culture-dependent identification” means methods of determining microbial presence using culturing techniques. For example, culturing a retentate sample in LB broth may be used to determine bacterial presence in the sample. Other culture methods are also envisioned, such as selective and differential media systems like MacConkey's agar and mEI agar. Additional selective culturing conditions or inclusion of additional techniques after culturing, such as NASBA, sequencing, PCR, or RFLP haplotyping, may be used to identify particular microbes.

As used herein, “selective staining” means identification using stains which target unique cellular structures, nucleotides, or peptides. Stains can be selected for detection of total protein, for distinct protein domains, total DNA or RNA, and distinct DNA or RNA domains. For example, selective staining of distinct DNA sequences using fluorescence in situ hybridization (FISH) allows for the discrimination and identification of microbes. Other stains include Gram staining, Ziehl-Neelsen staining, fluorescent dyes, and differential stains for distinct structures, like flagella.

As used herein, “shape-based identification” means a detection method which uses the shape of the target microorganism, or the shape of a structural region on the target microorganism, to detect the presence of the target. Exemplary methods include immunoassays, such as ELISA or fluorescent labeling for microscopy.

As used herein, “immune-based identification” or “immune-based assay” means a detection method utilizing antibodies or antibody mimics. The antibodies selectively bind to target molecules, allowing for selective staining, purification, isolation, or other means. “Antibody” includes all products derived, or derivable, from antibodies or antibody genes, including without limiting the scope of the invention natural antibodies, antibody fragments, antibody derivatives, genetically-engineered antibodies, or combinations thereof. Antibody mimics include molecularly imprinted polymers, aptamers, peptides, or combinations thereof. Examples may include ELISA, fluorescent antibody detection, RIA, Western blot, and immuno-electron microscopy.

As used herein, “nucleic acid-based identification” means an assay which uses oligonucleotide sequences to selectively hybridize to target sequences. The “oligonucleotide” is a nucleic acid sequence isolated from a natural source, synthetically manufactured, produced from restriction enzyme digestion, or genetically engineered. The oligonucleotide may be suspended in a solution or attached to a support, such as covalently attached to a support. Exemplary nucleic acid-based identification assays include PCR, RAPD-PCR, nucleic acid probes, NASBA, plasmid fingerprinting, and sequencing.

As used herein, “sequence-based identification” means an assay using the sequence of component molecules making up a larger molecule or polymer to identify microorganisms. The detection assay may use sequencing of oligonucleotides peptides, or other biological polymers. Exemplary detection methods include solid phase and liquid phase arrays, Edman degradation with HPLC and liquid chromatography-mass spectrometry (LC-MS) for proteins, and the Sanger and Maxam-Gilbert methods for nucleic acids.

As used herein, “carbohydrate-based identification” is a detection method using the characterization of carbohydrate molecules or fragments by methods such as gas chromatography-mass spectrometry (GC-MS) with selected ion monitoring (SIM) or GC-tandem mass spectrometry (GC-MS-MS). A “carbohydrate” is a molecule having a general formula Cₓ(H₂O)_{y}, such as monosaccharides, disaccharides, oligosaccharides, and polysaccharides. Examples of carbohydrate-based identification are known in the art (Gilbart, et al., Carbohydrate profiling of bacteria by gas chromatography-mass spectrometry: chemical derivatization and analytical pyrolysis. Eur J Clin Microbiol. 1987 December; 6(6):715-23; Fox, Carbohydrate profiling of bacteria by gas chromatography-mass spectrometry and their trace detection in complex matrices by gas chromatography-tandem mass spectrometry. J Chromatogr A. 1999 May; 843(1-2):287-300).

As used herein, “fatty acid-based identification” is a detection method using the characterization of fatty acids by methods such as GLC or electrospray time-of-flight mass spectrometry (ESI-TOF MS). Non-limiting exemplary methods are known in the art (Stahl and Klug, Characterization and differentiation of filamentous fungi based on fatty acid composition. Appl Environ Microbiol. 1996 November; 62(11):4136-46; Diogo, et al., Usefulness of fatty acid composition for differentiation of Legionella species. J Clin Microbiol. 1999 July; 37(7):2248-54).

As used herein, “size-based identification” is a detection method using the size of a microbe or molecule isolated from a microbe to identify the microbe. Examples are gel electrophoresis, like agarose, SDS-PAGE, and protein gel electrophoresis, and chromatography, like gas-liquid chromatography.

As used herein, “mass-selective identification” is any method that uses the mass or mass and charge of fragments and molecules from a particular microbe to detect it in a sample. Exemplary mass-selective methods include mass spectrometry, MALDI, MALDI-TOF, ESI-MS, and similar systems.

As used herein, “charge-selective identification is any method that uses the charge on a microbe or molecule or fragment from a microbe to detect it in a sample.

In the following detailed description of the preferred embodiments, reference is made to the accompanying drawings, which form a part hereof, and within which are shown by way of illustration individual embodiments by which the invention may be practiced. It is to be understood that other embodiments may be utilized and structural changes may be made without departing from the scope of the invention.

The concentration system filters particulate matter that is larger than the pore size of the filter from a liquid stream. Particulate matter collects within the hollow cores of the filter fibers, and the matter is subsequently recovered by backflushing the filter with a predetermined volume of recovery fluid such as water, buffer or other solution. The system may be backflushed by running the recovery fluid in an opposing flow from the test fluid. The concentration of collected particulate matter (e.g., bacteria, viruses, toxins) is much greater in the recovered retentate than in the original water source. The retentate may be directed to a detection system for detection and identification of its constituents. The inventive system also optionally includes a cleaning function that washes the system after every concentration cycle and readies the system to start a new cycle. The entire process is automated and controlled by a programmable logic controller. The programmable logic controller can be equipped with software tailored to the system's intended use. Examples of programmable variables include, inter alia, test fluid collection time, purge delay and time, volume of backflush solution, cleaning time and delivery of the concentrated sample to a biosensor for analysis.

The present invention is useful in concentrating particulates, including analytes of interest, in a filter during continuous flow of a test solution, such as water. A non-limiting example is the concentration of bacteria, such as Escherichia coli, from a municipal water source. The individual embodiments of the invention described herein use a tubular filter; however, any filter possessing the ability to collect test particulates is envisioned by this invention, such as a flat, rectangular filter or a box-shaped filter. By way of example only, one embodiment uses a filter produced by Fresenius Medical Care North America (Lexington, Mass.) that is amenable to processing large volumes of water. The inventive system utilizes backflushing to recover and concentrate the analyte(s) of interest that are captured by the filter. In such instances, it is advantageous to utilize a filter possessing backflush capabilities. Backflushing of the filter removes particulate matter collected on the interior of the filter fibers. Backflushing also accommodates periodic cleaning of the filter, thereby extending filter use.

The system includes a series of pumps adapted for controlling the flow of fluids throughout the system. For example, pumps are utilized in controlling the flow of recovery fluid and/or air flushes. The pumps used in the system may be any type useful in handling the required flow rates and pressures needed for analyzing water sources, such as municipal water supplies. In specific embodiments, positive displacement pumps are used, such as bellows, double-diaphragm, flexible impeller, rotary lobe, rotary vane, oscillating, piston, progressing cavity, and peristaltic pumps. Without limiting the scope of the invention, exemplary pumps are metering pumps, such as those described in Table 1.

TABLE 1
A listing of pumps, with flow rates and pressures.

| | Max. Flow rates | | Maximum | | Particulate | |
|---|---|---|---|---|---|---|
| Pump type | GPM | L/min | pressure | | matter | Notes/Advantages |
| Bellows | 0.008 to 26.4 | 0.03 to 100 | 73 | psi | Yes | Useful for liquids |
| | | | | | | or gases. |
| Diaphragm | 0.003 to 5.2 | 0.03 to 100 | 300 | psi | Not recom. | Ideal for high-accuracy |
| | | | | | | applications. |
| Air- | 5.0 to 225 | 19.0 to 851 | 125 | psi | Yes | Use for particulate-laden |
| operated | | | | | | fluids and where electric |
| diaphragm | | | | | | is not available. |
| Double- | 1.0 to 4.0 | 3.79 to 15.1 | 95 | psi | Yes | Use for particulate-laden |
| diaphragm | | | | | | fluids |
| Peristaltic | 0.00002 to 1.43 | 0.00008 to 45 | 125 | psi | Yes | Non-contaminating; |
| (tubing) | | | | | | available in |
| | | | | | | a wide variety materials. |
| Piston | 0.004 to 107 | 0.015 to 405 | 5000 | psi | No | High pressure and accuracy. |
| Syringe | 0.002 to 0.04 | 0.008 to 0.15 | 40 | psi | No | Low flow rates at high |
| | | | | | | pressures. |
| Nutating | 0.25 to 1.0 | 0.95 to 3.8 | 15 | psi | Yes | Polytetrafluoroethylene |
| disc | | | | | | wetted parts; positive |
| | | | | | | displacement |
| Flexible | 3.8 to 50.0 | 14.4 to 189 | 60 | psi | No | |
| impeller | | | | | | |
| Flexible | 1.0 to 10.0 | 3.8 to 37.8 | 50 | psi | Yes | Pulseless pumping uses no |
| liner | | | | | | seals, can run dry. |
| Progressing | 0.5 to 13 | 1.9 to 49 | 100 | psi | Yes | Pulseless flow allows for |
| cavity | | | | | | particulate-laden fluids. |
| Rotary | 0.75 to 4.3 | 2.8 to 16.3 | 240 | psi | No | High-pressure capabilities; |
| vane | | | | | | low shear. |

Information Collected from Cole-Parmer Instrument Company; Vernon Hills, Ill.

The system also utilizes valves to control and direct the flow of fluid through the system. The valves used in the system may be any type adapted to handle the pressures needed for analyzing water sources, such as municipal water supplies. Further, the valves should have quick response times and minimal upkeep requirements. Valves in the present invention are used to control flow of test fluid, recovery fluid, air and cleaning solution. Some non-limiting examples include butterfly valves, trunnions, mounted ball valves, plug valves, globe valves, gate valves, angle seat piston valves, angle valves, ceramic disc valves, piston valves, pinch valves, and mass flow controllers. It is noted that electronically-actuated valves, such as mass flow controllers, are very useful in the present invention as they are robust, cover a wider range of uses and extend lifetime. In general, preferred valves are selected based on small size, light weight, reliability, and the ability to withstand at least 100 PSI. Additionally, the use of electronically-actuated ball valves for test fluid input and the outflow of filtered test fluid permits increased flow rate of test fluid through the system and drastically decreases the time needed to filter a given volume of test fluid.

Advantageously, the test fluid pressure is monitored during the concentration process using any pressure-sensitive device known in the art. Some embodiments use transducers to monitor pressure of the test fluid either before the filter, after the filter, or at both locations, before and after the test fluid is filtered. Transducers disposed both before and after the filter permit pressure differences of flow through the filter fibers to be monitored. Inclusion of a flow meter permits monitoring of flow rate and total filtered volume of test fluid. Non-limiting alternatives include piezo-resistive pressure sensor, piezo-resistive pressure transducer, miniature cylindrical pressure transducer, silicon strain gauge pressure transducer, pressure transmitter, digital pressure gauge, and analog pressure gauge. The pressure gauge located after the filter is placed in a position that permits measurement of the backflush pressure as well as the post-filter forward flow pressure. These additions provide for better process monitoring and feedback to control software to permit processes to be controlled based on pressure and flow parameters. For example, if control software determined that pre-filter pressure and flow rate had fallen outside set parameters, the filtration process would be terminated by the program.

The Backflush subsystem permits either a gravity drain of the fiber cores, an air-flush of the fiber cores or a liquid backflush using a pre-chosen solution to remove particulate material trapped within the filter. The gravity drain function is accomplished by opening valves located on the top and bottom of the supports that hold the filter housing in position. While other mechanisms can be used, the use of a pump, such as a metering pump, for backflush sequences increases flexibility in developing effective backflush sequences and improves recovery for a large range of filter types and sizes. Where the concentrator has all three systems—gravity, air, and liquid—the sequence of the three backflush options are programmed into, and controlled by, the PLC. Particulate matter released from the filter passes through the sample-drain and is collected in a collection vessel. Material in the collection vessel may then be delivered to a biosensor or other detection method, for detection and identification of particulates. The inventive method is not limited by any one sequence of events; however, clearing liquid from the fiber cores of the filter and using air before backflushing with a recovery fluid enhances the efficiency of the backflush step. The recovered, concentrated retentate is then optionally sent to a detection system, where it may be analyzed for any number of factors, such as microbial constituents present and their relative number and/or presence of other particulate substances that might be harmful. A variety of different types of detection systems are compatible with the sample recovered during backflush. Exemplary detection systems may include methods such as culture-dependent, biochemical, immunoassay, nucleic acid-based, mass-charge and ion-detecting, magnetic, optical and spectral identification methods. Non-limiting examples include culture-dependent optical methods, such as those described by Abbas, et al. (U.S. Pat. No. 5,223,402), Powers, et al. (U.S. Pat. No. 6,750,006), Edberg (U.S. Pat. No. 6,783,950) and Enterolert (IDEXX, Westbrook, Me.); optical differential staining, such as described by Alford, et al. (U.S. application Ser. No. 12/106,887), Cools et al., (Solid phase cytometry as a tool to detect viable but non-culturable cells of Campylobacter jejuni. Journal of Microbiological Methods, 2005, 63(2): 107-114) and EasyStain (BTF, Sydney, Australia); biochemical methods such as described by Devic, et al. (Detection of Anatoxin-a(s) in Environmental Samples of Cyanobacteria by Using a Biosensor with Engineered Acetylcholinesterases. Applied Environmental Microbiology, 2002, 68: 4102-4106), Aathithan, et al. (Diagnosis of Bacteriuria by Detection of Volatile Organic Compounds in Urine Using an Automated Headspace Analyzer with Multiple Conducting Polymer Sensors. Journal of Clinical Microbiology, 2001, 39: 2590-2593) and Mittelmann, et al. (Amperometric Quantification of Total Coliforms and Specific Detection of Escherichia coli. Analytical Chemistry, 2002, 74:903-907); immunoassay and similar shape-based capture methods such as described by Hunter, et al. (Rapid detection and identification of bacterial pathogens by using an ATP biolumninescence immunoassay. Journal of Food Protection, 2010, 73:739-46), Ligler et al. (The Array Biosensor: Portable, Automated Systems. Analytical Sciences, 2010, 23: 5-10) and Maher et al (U.S. Pat. No. 7,749,775); nucleic acid detection, such as those described by Kulichenko, et al. (Improvement of a method for detecting of strains of the plague microbe using polymerase chain reaction. Genetika, 1994, 30(2): 167-71), Blais et al. (A nucleic acid sequence-based amplification (NASBA) system for Listeria monocytogenes and simple method for detection of amplimers. Biotechnology Techniques, 1996, 10(3): 189-194), Leskinen et al., (Hollow-fiber ultrafiltration and PCR detection of human-associated genetic markers from various types of surface water in Florida. Applied Environmental Microbiology, 2010, 76(12): 4116-7) and Baeumner et al. (RNA biosensor for the rapid detection of viable Escherichia coli in drinking water. Biosensors and Bioelectronics, 2003, 18(4): 405-413) or mass spectrometric, so-called reagentless, label-free and spectral detection, systems such as those described by Snyder et al. (Correlation of Mass Spectrometry Identified Bacterial Biomarkers from a Fielded Pyrolysis-Gas Chromatography-Ion Mobility Spectrometry Biodetector with the Microbiological Gram Stain Classification Scheme Analytical Chemistry, 2004, 76:6492-6499), Grow et al. (New biochip technology for label-free detection of pathogens and their toxins. Journal of Microbiological Methods, 2003, 53:221-233) and Alupoaei, et al. (Quantitative spectroscopy analysis of prokaryotic cells: vegetative cells and spores. Biosensors and Bioelectronics, 2004, 19:893-903). Some of these detection systems may be in the form of automated detectors that can be connected to the retentate sample produced by the inventive system to analyze the sample without human intervention. A wide range of these automated sensors exist that combine various biological recognition elements with a variety of available transducers (Lazcka, et al., Pathogen detection: a perspective of traditional methods and biosensors. Biosensors and Bioelectronics. 2007, 22(7): 1205-1217; Velusamy et al., An overview of foodborne pathogen detection: in the perspective of biosensors. Biotechnology Advances. 2010, 28(2): 232-254; Emanuel and Fruhey, Market Survey: Biological detectors 2007 Ed.). It is also envisioned that the retentate sample taken from the present invention may be further concentrated prior to analysis, such as by a detection method described above. Non-limiting examples of a concentrators which may be used in concert with the present invention include the InnovaPrep concentrator (Innovaprep LLC, Drexal, Mo.), immune-magnetic beads, selective columns, and sample preparation methods like automated modules for sample prep for PCR (Belgrader, P.; Elkin, C. J.; Brown, S. B.; Nasarabadi, S. N.; Langlois, R. G.; Milanovich, F. P.; Colston, B. W., Jr.; Marshall, G. D. Anal. Chem. 2003, 75, 3446-3450) or microchip gel electrophoresis (Stachowiak, J. C., Shugard, E. E., Mosier, B. P., Renzi, R. F., Caton, P. F., Ferko, S. M., Van de Vreugde, J. L., Yee, D. D., Haroldsen, B. L., and VanderNoot, V. A. Anal. Chem. 2007, 79, 5763-5770).

The following represents an illustrative device developed based on the methods of the inventive system. This example represents only one filtration device that permits concentration of particles, including microorganisms, from the test fluid according to the inventive method. It is to be understood that the embodiments described below are specific variants of the invention, and that changes from these specific variants, such as replacing the types of valves or pumps, is within the scope of the present disclosure.

### Example 1

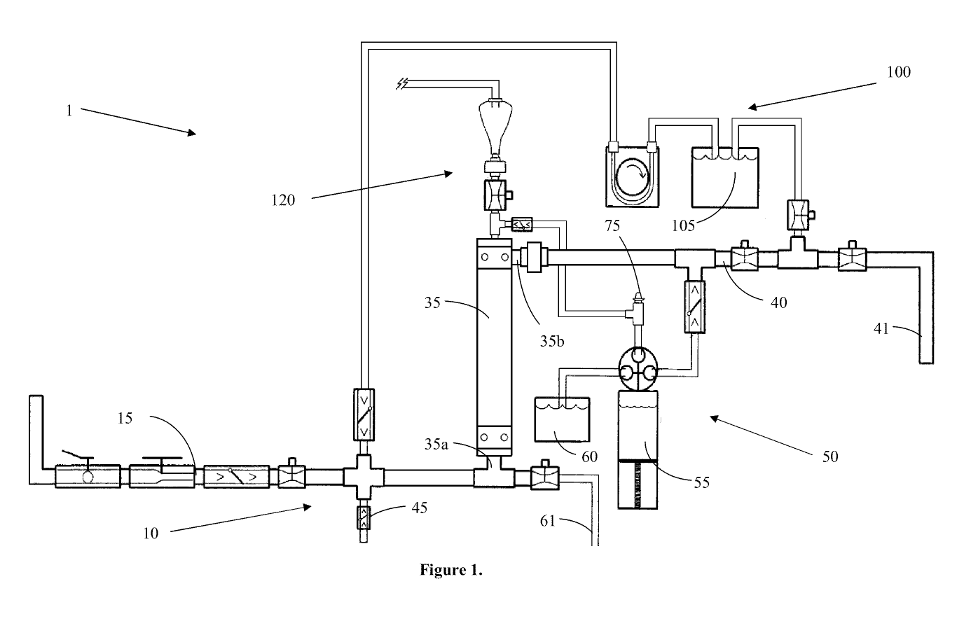

Referring now to the figures, FIG. 1 shows a schematic view of an illustrative device. Automated Concentration System (ACS) 1 comprises forward-flow concentration subsystem 10, with a backflush compatible filter, backflush subsystem 50, cleaning subsystem 100 and purge subsystem 120. The filter in the present example is a tubular hollow fiber filter having an interior, such as a core, and an exterior outside of the fiber walls or housing side.

### Forward-Flow Concentration Subsystem (FFC)

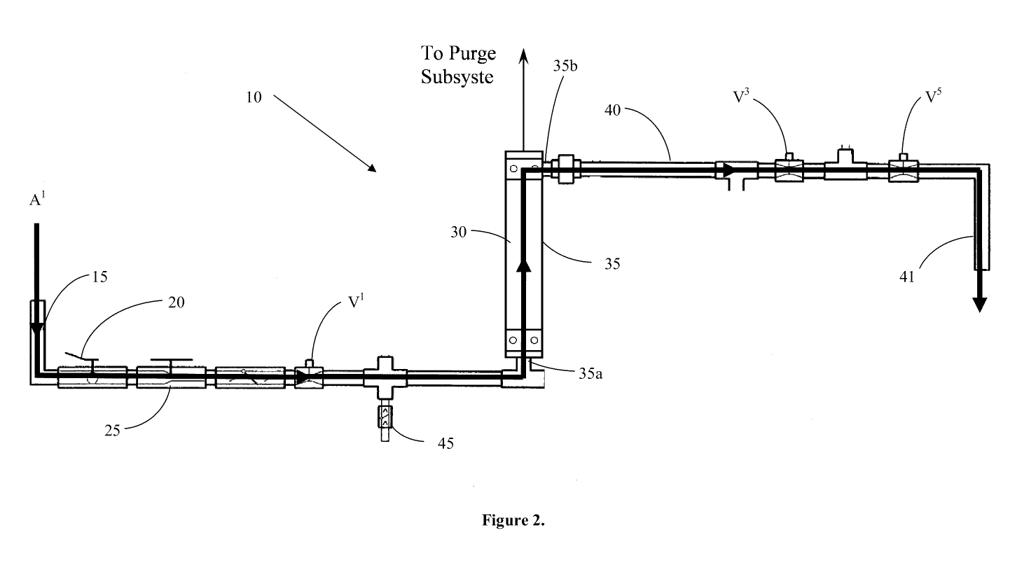

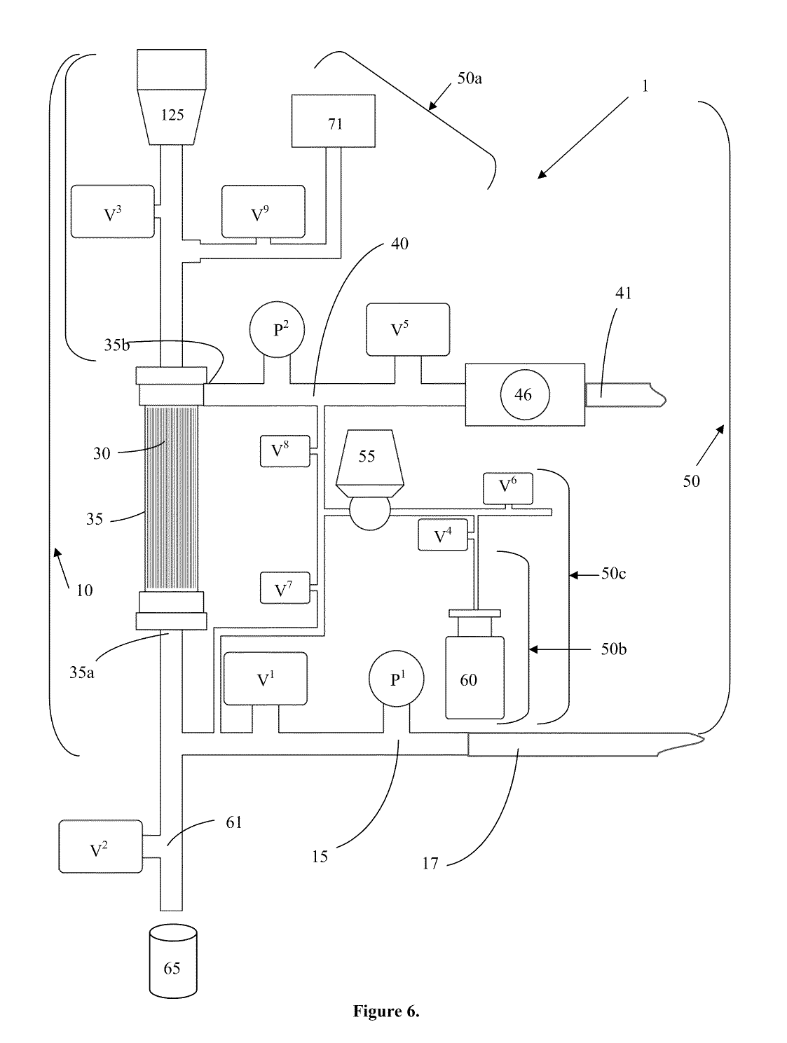

Forward-flow concentration subsystem (FFC) 10, shown in FIG. 2, includes filter housing 35, with a fluid input 35a and a fluid output 35b. Filter housing 35 contains a filter 30 comprised of hollow, tubular fibers. Fluid input 35a is disposed toward the center of filter housing 35, to permit test fluid to flow into the interior of the fibers of filter 30. Fluid input 35a is attached to source line 15, thereby directing the test fluid flow from a fluid source through FFC 10 as indicated by arrow A¹ seen in FIG. 2. An optional pressure monitor, P¹ shown in FIG. 6, may be installed before filter 30 to measure pressure of test fluid into the filter. For example, a pressure gauge P¹ may be installed before mass control valve V¹ seen in FIG. 6. Source line 15 may also include optional spiking port 45, seen in FIG. 1, thereby allowing a user to introduce additional materials into the unfiltered test fluid. For example, on occasion it may be necessary to test the efficiency of the device. In such situations, the user may inject a microbe into the injection port, and allow the concentrator to cycle through its operation. Upon completion, the injected material is tested to confirm the concentrator is performing adequately. Insertion or attachment of other functional parts into source line 15 is envisioned. As one example, in applications involving high turbidity water, an optional size-exclusion prefilter may be installed to remove large particulates that could clog filter 30. Other types of liquid pre-treatment that could be inserted into source line 15 include sieves, depth filters, ion exchange filters, materials/filters for chemical adsorption or absorption, and/or magnets. An optional electronically-controlled ball valve is mounted before the prefilter and/or after the prefilter. As another example, optional flexible source tubing 17, seen in FIG. 6, may be attached to source line 15 to allow for insertion of additional functional parts or to extend the reach of source line 15 to a more distant source fluid. Other functional parts may include one-way valves to prevent backflow to the source water, injection ports for spiking and introducing materials to the line further upstream from the filter 30, at least one pump adapted to move test fluid into the lines leading to the filter, pressure monitoring and pressure regulating devices to prevent input pressure from exceeding 100 PSI, and mass controller valves. Mass flow control valve V¹ is used to control the flow of test fluid through source line 15 to filter 30 as seen in FIG. 2.

The inventive system uses dead-end flow filtration to filter the test fluid. Test fluid is directed into the interior of the hollow fibers of filter 30, and travels parallel to the fiber walls, until it reaches the distal end of the filter, which is closed. The test fluid cannot recirculate, and is forced through the pores in the walls of the fibers. Liquid and molecules smaller than the pore size travel through the pores, with any larger molecules being trapped within the fiber cores, as seen in FIG. 2. Thus, any particulates, including microorganisms and biological compounds, larger than the pore size of the filter are retained within the fiber cores and all other material passes to the exterior space of filter housing 35. Accordingly, the pore size of the filter can be used to select for a specific size range of pathogen or particulate matter that is less than the pore size of the filter. Fluid output 35b is disposed on the outer wall of filter housing 35, thereby collecting test fluid from the outer walls of the hollow fibers of filter 30 that accumulates in the filter housing 35 after filtration of the test fluid. The fluid input and output are disposed to allow test fluid flow A¹ to move from the interior of the hollow fibers to the outer walls of the fibers.

Fluid filtrate line 40 is attached to fluid output 35b and directs the filtered test fluid to drain line 41. A pressure monitor P² and a flow meter 46, seen in FIG. 6, are optionally attached to fluid filtrate line 40. The pressure monitor P² permits monitoring of fluid pressure after filter 30 during filtration and, together with pressure monitor P¹, helps to determine when the filter 30 is too fouled to continue filtration. The flow meter 46 permits measurement of flow rate, which also helps determine when the filter 30 is too fouled to continue filtration. It also monitors total volume filtered, which can be used to determine how much the process has concentrated particulates from the test fluid.

### Backflush Subsystem

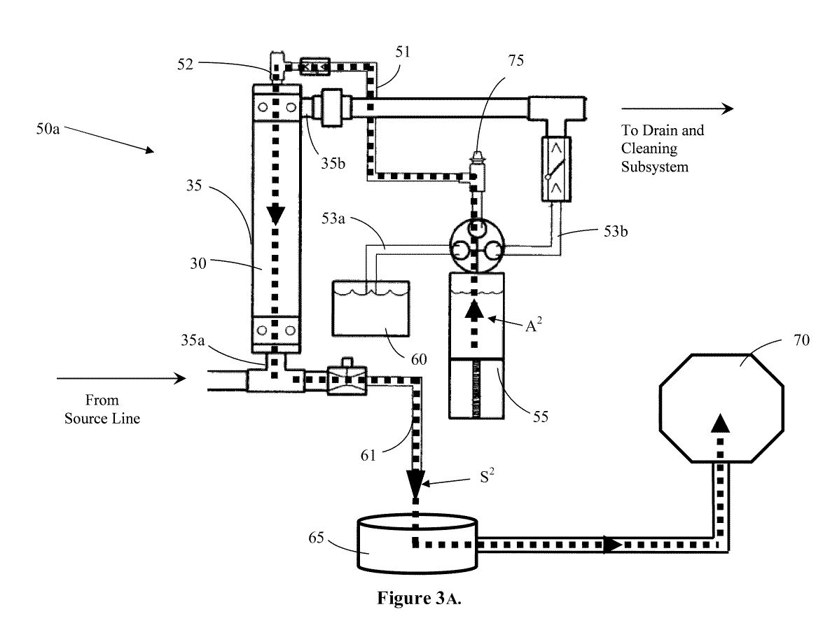

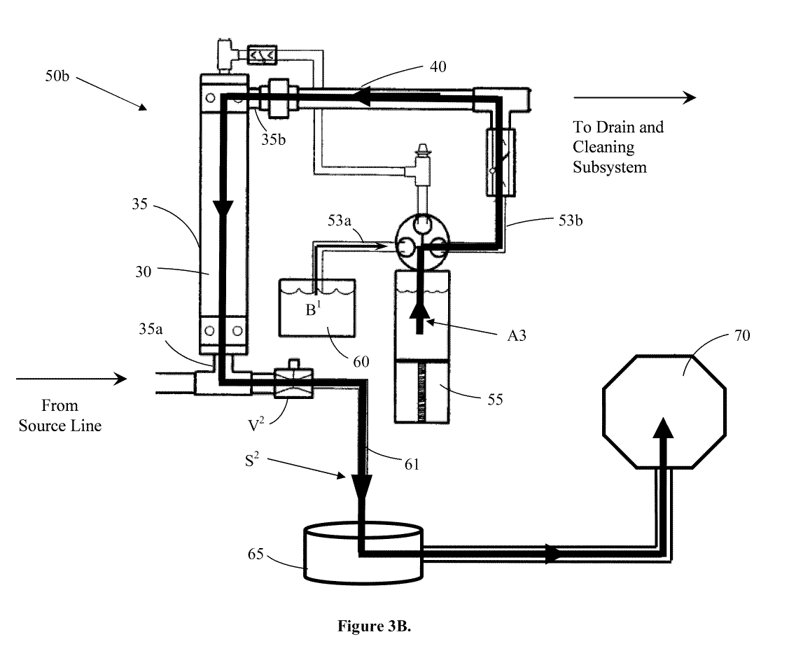

Backflush subsystem 50 further comprises air-backflush subsystem 50a, seen in FIG. 3A, and liquid-backflush subsystem 50b, seen in FIG. 3B. The programmable logic controller (PLC) initiates backflush subsystem 50 after a predetermined amount of water passes through filter 30. The PLC turns off water flow to filter 30 prior to engaging a backflush sequence. Backflush subsystem 50 permits either a gravity drain of the fiber cores, an air-flush of the fiber cores, as seen in FIG. 3A, or a liquid backflush through the fiber walls into the fiber cores using a pre-chosen solution, as seen in FIG. 3B, to remove particulate material trapped within the fiber cores of filter 30. In this embodiment, the system utilizes both an air-backflush subsystem 50a and a liquid-backflush subsystem 50b.

The backflush subsystem comprises backflush pump 55 and air-backflush line 51 connected to the output of backflush pump 55. Disposed along air-backflush line 51 is air-valve 75 to allow air-backflush subsystem 50a to collect ambient air for use in the air-backflush. Optionally, air-valve 75 may also include an air filter to prevent introduction of particulates to the system during air uptake into air-backflush line 51. Air-backflush line 51 connects to filter housing 35 at air-backflush input 52. As seen in FIG. 3A, air-backflush input 52 is disposed on filter housing 35 such that the input of backflush air is introduced to the interior of the fibers of filter 30.

Air-backflush subsystem 50a is shown using ambient air to flush the system, however any fluid can be incorporated and the selection of an appropriate gas will require an analysis of the intended use of the system. The PLC initiates an air-backflush sequence starting pump 55, which then draws air through air-valve 75. The air then travels under pressure along path of travel A² through filter 30, thereby removing liquid from the fiber cores along with some particulate matter trapped therein. The backflush sample continues through fluid input 35a, which provides input of the original test fluid to filter 30, and also allows backflush fluids to be removed from the filter and collected. The backflush sample moves along path of travel A² through sample-drain 61 into collection vessel 65. The sample in collection vessel 65 can be analyzed using a variety of standard and rapid methods including biosensors. Non-limiting examples of useful detection methods are provided above, and include methods such as culture-dependent, biochemical, selective staining, immunoassay and other selective capture, nucleic acid-based capture, carbohydrate-selective, fatty acid selective, mass-, charge- and/or ion-selective, optical and spectral identification methods. Exemplary detection methods include ELISA, bioluminescence and electrochemiluminescence, growth in/on selective or differential media, optical differential staining, fluorescent antibody tagging; nucleic acid tagging, nucleic acid sequence-based amplification (NASBA), polymerase chain reaction (PCR), ion mobility spectroscopy (IMS), and mass spectrometry. FIGS. 3A and 3B show one example in which the sample is directed from vessel 65 to the optional biosensor 70 responsive to a signal from the PLC. Parameters governing delivery to the biosensor are varied but can include time and/or volume. Useful biosensors are known and will be apparent to one skilled in the art considering factors such as the particulate matter being analyzed and the intended use of the system.

Liquid backflush subsystem 50b also utilizes backflush pump 55, seen in FIG. 3B. However, the liquid backflush subsystem uses independent liquid-backflush lines 53a and 53b, as seen in FIG. 3B. Solution reservoir 60 is connected to backflush pump 55 by liquid-backflush line 53a, thereby allowing backflush liquid to be pressurized. Liquid-backflush line 53b further connects the liquid backflush system to fluid filtrate line 40 and to filter 30 via fluid output 35b.

A preselected backflush solution is stored in solution reservoir 60. Solution reservoir 60 can be filled with any liquid, the selection of which may vary depending on the system's intended use. Commonly, reservoir 60 will be filled with a predetermined quantity of water, buffer or other solution. The PLC activates the liquid backflush subsystem, thereby initiating backflush pump 55, which draws liquid out of solution reservoir 60 through liquid-backflush line 53a along path B¹ and pressurizes the liquid. The pressurize backflush liquid then flows through to liquid-backflush line 53b along path A³ to fluid filtrate line 40 and enters filter housing 35 through fluid output 35b. The backflush solution flows from the exterior walls of filter 30 to the interior cores, thereby removing any collected particulate matter trapped therein to form a sample. The sample continues along path of travel S² through sample-drain 61 into collection vessel 65. The sample can be directed from vessel 65 and subsequently tested using biosensor 70 or any detection technique known in the art. Parameters governing delivery to the biosensor are varied but can include time and/or volume.

### Cleaning Subsystem

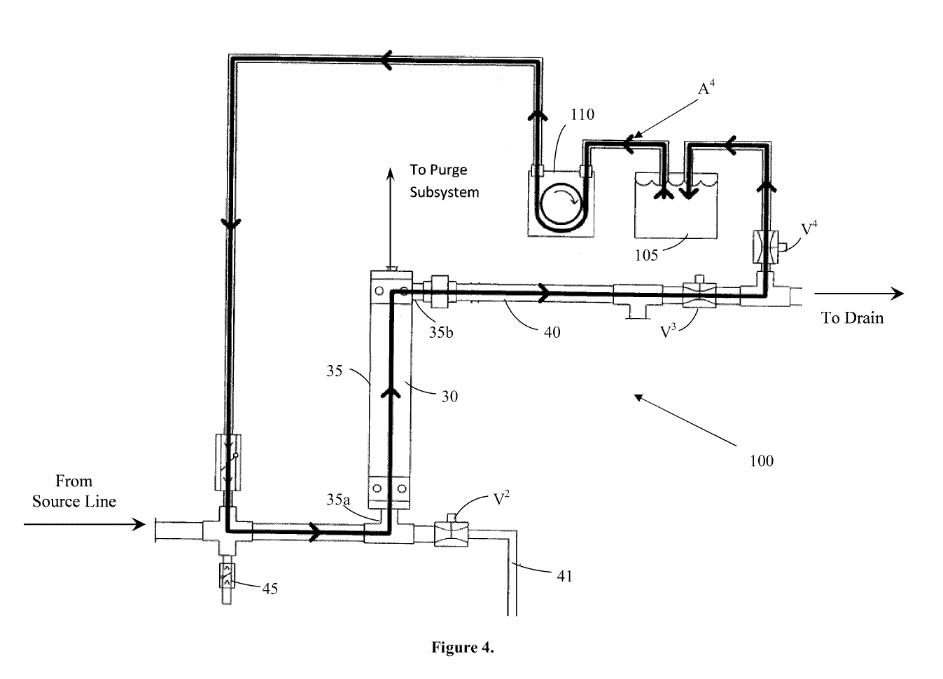

The cleaning sequence initiates responsive to a signal from the PLC once the particulate matter in filter 30 has been backflushed into the collection vessel. Cleaning solution reservoir 105, seen in FIG. 4 may optionally incorporate a precision temperature control device, where the cleaning solution is heated prior to the cleaning step. In this illustrative embodiment reservoir 105 holds up to 5 liters of cleaning solution at a user-determined temperature. Alternatively, reservoir 105 contains cleaning solution at ambient temperature. Cleaning subsystem 100 circulates the cleaning solution through filter 30 in the forward flow path of travel (A⁴), as seen in FIG. 4. A cleaning cycle is completed when the cleaning solution returns to reservoir 105, but multiple cleaning cycles can be incorporated into a single cleaning sequence. The type of solution, cleaning temperature and length of cleaning are determined by the user. The cleaning solution is removed from filter 30 and system lines by a combination of forward flow and backflush events initiated by the PLC.

A new forward flow concentration cycle can be started upon the successful completion of the cleaning sequence. If desired, two or more units can be linked to the source flow and collection alternated between the two units. Redundant use of the inventive system ensures that one unit is operational while the other is being cleaned thereby eliminating gaps in collection.

### Purge Subsystem

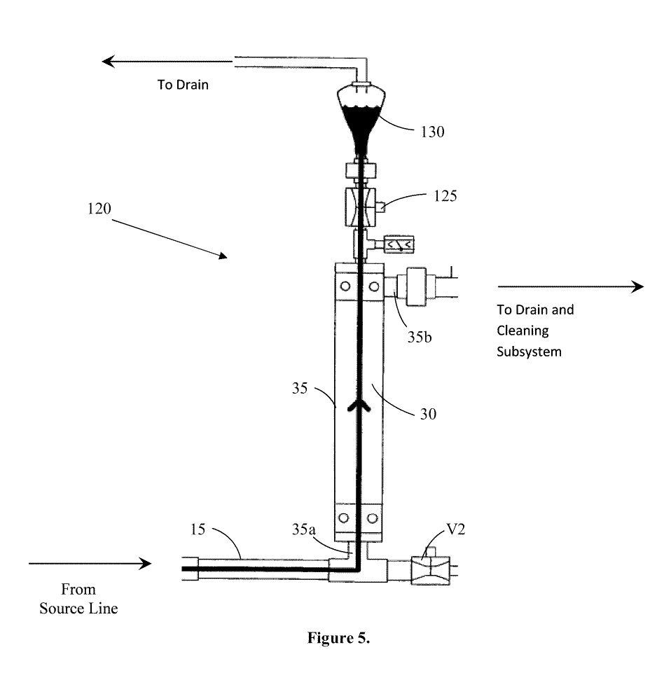

Purge subsystem 120, seen in FIG. 5, comprises purge valve 125 and purge reservoir 130. Purge valve 125 and purge reservoir 130 are optimally positioned at the top of filter housing 35 to permit the escape of any air or gas that has collected within filter 30 during the initiation of forward flow functions such as filtration and cleaning. This safety feature prevents flow shutdown due to air pressure buildup at the outflow point of filter housing 35. Pressure gauges located on the inlet and outlet of filter housing 35 permit the pressure on both sides of the filter 30 to be monitored.

### Programmable Logic Controller (PLC)

Automation of the inventive system is possible with the use of a programmable logic controller (PLC). The term programmable logic controller (or PLC) as used herein is any device used for the automation of the disclosed system. While the PLC usually will incorporate a microprocessor, devices relying on mechanical control (i.e. timers) are also contemplated. In a preferred embodiment the PLC remains in electronic communication with the constituent elements of the system, including sensors, valves, solenoids, pumps, gauges and actuators. The input/output arrangements necessary to practice the invention may be built into a simple PLC, or the PLC may have external input/ouput modules attached to a proprietary computer network that plugs into the PLC. Although the current system is optimized for automation, manual operation is also envisioned.

In a preferred embodiment, the PLC is equipped with software that provides an interface for control of forward flow (concentration) time and volume, purge delay and length, forward flow of liquid and air, interior filter drain time, number of air flushes and air flush time, number of liquid backflushes and backflush time, sequence of air and liquid backflush events, cleaning solution circulation time and cleaning solution flush sequence and time. In certain embodiments of the invention, the PLC includes an interface for control of an optional test fluid delivery pump. In such embodiments using the test fluid delivery pump, the pump directs the flow of test fluid into the system input. The PLC may also include in specific embodiments, an interface for connecting to an optional automated biosensor for detection of analytes in the retentate sample. A system diagram incorporated into the user interface can provide feedback on flow paths during operation. Controls may also be provided to configure the system for introduction of a sample to test the operation of the system. An assay recipe program directs the sequence of concentration steps. The recipe program includes a choice of standard concentration processes or provides flexibility by allowing the user to encode a different sequence, if desired, prior to initiating the concentration process.

The PLC controls flow through the system by opening and closing valves, V¹ through V⁵, located at strategic points on the system. As noted, the valves may be any known in the art. In the cleaning sequence shown in FIG. 4, for example, the PLC would open solenoid valves V³ and V⁴ but close solenoid valves V′, V² and V⁵, seen in FIGS. 2, 4, and 5. A check valve can be incorporated to prevent the introduction of fluid into the backflush subsystem.

### Example II

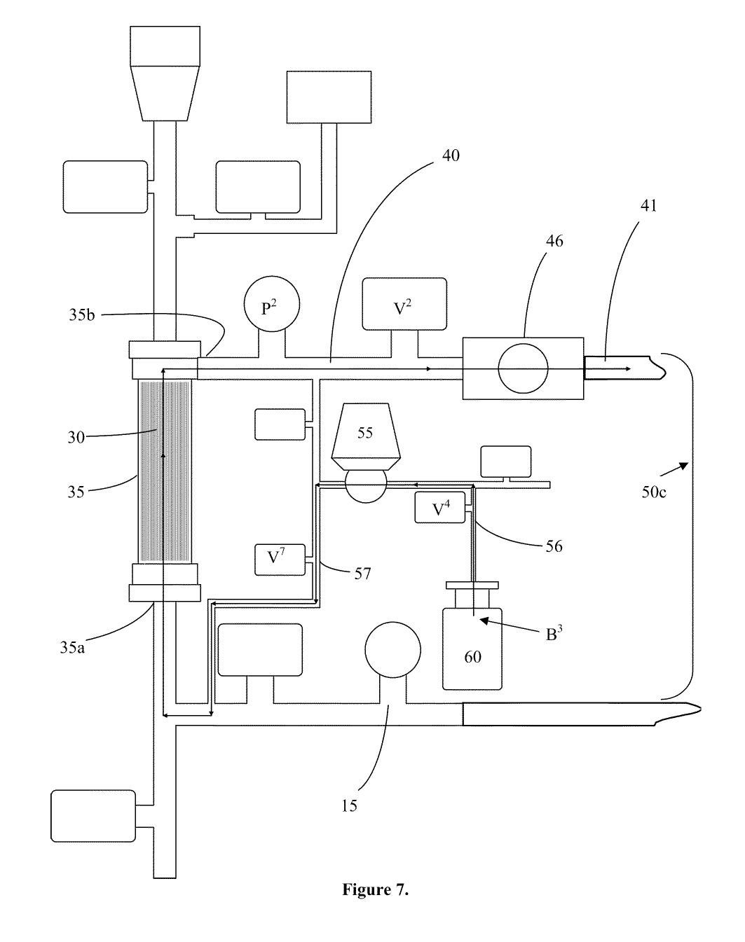

In addition to the concentrator in Example I, another embodiment of the concentrator, shown in FIG. 6, may include the capability to forward flow the recovery fluid, i.e. flow the recovery fluid in the same path of travel as test fluid, as seen in FIG. 7. Forward flow buffer line 57 is in fluid communication with source line 15, allowing the recovery fluid to enter filter housing 35 through fluid input 35a, leading into the fiber cores of filter 30. Mass control valve V⁷ controls the introduction of recovery fluid into source line 15, which permits the system to equilibrate filter 30 to the recovery fluid before the backflush sequence begins. In some embodiments, the recovery fluid is obtained from solution reservoir 60 using backflush pump 55. In this embodiment, liquid/air forward flow line 57 is in fluid communication with the output end of backflush pump 55 and forward flow buffer line 56 takes up buffer from solution reservoir 60 via backflush pump 55. Uptake of recovery fluid from solution reservoir 60 is controlled by mass control valve V⁴. Alternatively, an independent reservoir holds forward flow recovery fluid and is independent from the backflush system.

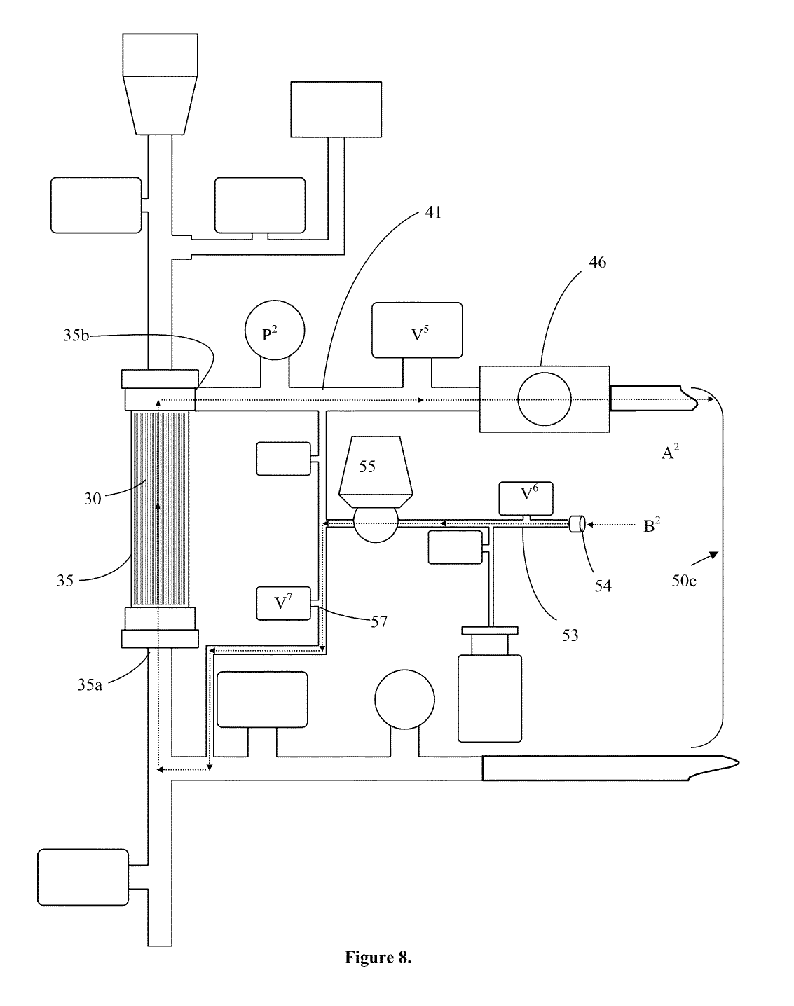

The concentrator may also include the capability to forward flow air through the filter, separately or in addition to the capability to forward flow recovery fluid, as shown in FIG. 8. Liquid/air forward flow line 57 is in fluid communication with source line 15, allowing the air to purge source line 15 and enter filter housing 35 through fluid input 35a, leading to the fiber cores of filter 30. Mass control valve V⁷ controls the introduction of air into source line 15. Once in filter housing 35, the air removes residual fluid from filter 30. In some embodiments, the air is supplied from ambient air drawn in through an air uptake line 53, seen in FIG. 8, which may be fitted with optional filter 54. In these embodiments liquid/air forward flow line 57 is connected to the output end of backflush pump 55, which pumps air through filter 54 into air uptake line 53 to liquid/air forward flow line 57 and into source line 15. Uptake of air through line 53 is controlled by mass control valve V⁶ and output of air to the filter 30 is controlled by mass control valve V⁷. Alternatively, an independent forward flow air pump or pressurized gas source may be used to introduce air into liquid/air forward flow line 57. The air enters the filter 30, thereby removing excess fluid from the filter.

The alternate embodiment seen in FIG. 6 incorporates changes in other system components that permit portability of the system by reducing size and weight. As one example, the purge reservoir 130 in Example 1 seen in FIG. 5 is replaced with purge valve 125 in FIG. 6. As another example, air backflush subsystem 50a in FIG. 3A has been replaced with compressed air source 71 in FIG. 6. Mass control valve V⁹ controls the entry to filter 30 in this embodiment.

### Example III

### Concentration of E. coli O157:H7 Spiked into Tap Water

A new 0.8 mm Norit hollow fiber filter or a used filter that had been soaking in 1% bisulfite solution preservative was used for each test. The filter was installed in the concentration system, the input line connected to a faucet and the filter rinsed with tap water to remove storage solution. The filter was then backflushed with distilled water. Prior to spiking, water was run through the filter in the forward direction for 5-7 minutes and the pressure before and after the filter and flow rate were measured. When a previously used and cleaned filter was used in a test, blank water samples were collected and recovered before each test and after each test to confirm that no residual test organisms carried over between experiments.

For each test, E. coli O157:H7 cells labeled with green fluorescent protein (GFP) were diluted into a syringe with 10 ml of distilled water and spiked into the tap water flowing into the concentration system using an injection port integrated into flexible tubing connecting the faucet to the inlet of the concentration system. Also integrated into the tubing between the spike port and the concentrator inlet was a mixing chamber to more closely approximate the dilution that might occur in natural contamination events. Spikes were followed by 10 additional milliliters of water to rinse all organisms through the spike port and into the line leading to the concentrator. Tap water was sampled for 20 minutes and pressures before and after the filter and flow rate were monitored during filtration. After filtration was stopped, recovery of E. coli O157:H7-GFP cells collected within the filter was initiated by first injecting recovery fluid, 0.1 M sodium phosphate buffer with 0.01% added sodium polyphosphate (PB+NaPP), into the filter using a port located just upstream of the filter inlet. Sufficient buffer was used to completely replace any tap water remaining in the filter and filter housing and the buffer was incubated with the filter for 10 minutes. A backflush sequence was initiated to remove collected particulates, including E. coli O157:H7-GFP cells, from the center of the filter fibers. The backflush sequence consisted of alternating both air backflushes through the center of the fibers only but in the reverse path of tap water entry to the filter fiber cores with PB+NaPP buffer backflushes from the outside of the fibers to the inside of the fibers. The retentate sample was collected into one or more containers through a port located at the bottom of the filter that connected to the center of the fibers. Both stock and retentate were enumerated using a counting chamber and epifluorescence microscopy. Recovery efficiency and concentration data for several experiments are presented in Table 2. The concentration system and all associated tubing were disinfected with a bleach solution and dechlorinated with a sodium thiosulfate solution at the end of each experiment. A PLC was used to control all steps in the concentration procedure except the forward flow of buffer into the filter, which was done manually.

TABLE 2
Data from experiments using the inventive method to recovery E. coli O157:H7 from tap water.

| | | | Sample | | |
|---|---|---|---|---|---|
| | Quantity spiked in | Volume | concentration | Retentate | |
| Experiment # | (CFU) | Filtered (L) | (CFU/ml) | Volume (ml) | % Recovery |
| 1 | 1.53 × 10⁶ | 32.4 | 4.66 × 10³ | 197.5 | 60.19 |
| 2 | 2.88 × 10⁶ | 35.3 | 2.34 × 10⁴ | 197.5 | 160.76 |
| 3 | 1.34 × 10⁷ | 35.7 | 6.16 × 10⁴ | 198.5 | 91.44 |
| 4 | 7.50 × 10⁷ | 24.9 | 2.87 × 10⁵ | 205.0 | 78.35 |
| Average ± S.D. | 2.32 × 10⁷ ± 3.49 × 10⁷ | 32.1 ± 5.0 | 9.42 × 10⁴ ± 1.31 × 10⁵ | 200.0 ± 3.61 | 97.7 ± 44.0 |

### Example IV

### Concentration of Bacillus atrophaeus Spores Spiked into Tap Water

A new Fresenius F200NR hollow fiber filter was installed into the concentration system prior to each test and the inlet line of the concentration system was connected to a faucet. For each test, B. atrophaeus spores were diluted into a syringe with 10 ml of distilled water and spiked into the tap water flowing into the concentration system using an injection port integrated into flexible tubing connecting the faucet to the inlet of the concentration system. Spikes were followed by 10 additional milliliters of water to rinse all organisms out of the spike port into the line leading to the concentrator. Tap water was sampled for 60-80 minutes and flow rate and the pressure before and after the filter were monitored during filtration. After filtration was stopped, B. atrophaeus spores collected on the filter were recovered by injecting recovery fluid, 0.1 M PB+NaPP, into the filter using a port located just upstream of the filter inlet. Sufficient buffer was used to completely replace any tap water remaining within the filter and filter housing and buffer was incubated with the filter for 10 minutes. A backflush sequence was initiated to remove collected particulates, including B. atrophaeus spores, from the center of the filter fibers. The backflush sequence consisted of alternating both air backflushes through the center of the fibers only but in the reverse path of tap water entry to the filter fiber cores with PB+NaPP buffer backflushes from the outside of the fibers to the inside of the fibers. The retentate sample was collected into one or more containers through a port located at the bottom of the filter that connected to the center of the fibers. B. atrophaeus spores were counted by filtering aliquots of the retentate through a 0.45 μm nitrocellulose filter and plating onto tryptic soy agar. B. atrophaeus colonies were differentiated by their morphology and color. Recovery efficiency and concentration data for several experiments are presented in Table 3. A PLC was used to control all steps in the concentration procedure except the forward flow of buffer into the filter, which was done manually.

TABLE 3
Data from experiments using the inventive method
to recover Bacillus atrophaeus from tap water.

| | | | Sample | | |
|---|---|---|---|---|---|
| | Quantity spiked in | Volume | concentration | Retentate | |
| Experiment # | (CFU) | Filtered (L) | (CFU/ml) | Volume (ml) | % Recovery |
| 1 | 2385.00 | 205.47 | 2.43 | 394.5 | 40.13 |
| 2 | 276.60 | 179.22 | 0.30 | 388 | 41.58 |
| 3 | 735.00 | 148.41 | 0.66 | 379 | 34.15 |
| 4 | 719.63 | 146.73 | 0.40 | 369.5 | 20.57 |
| 5 | 734.54 | 141.22 | 0.57 | 369.5 | 28.73 |
| 6 | 582.00 | 143.15 | 0.46 | 365 | 29.04 |
| 7 | 28620.00 | 142.99 | 36.51 | 380 | 48.48 |
| Average ± S.D. | 4864.68 ± 10497.22 | 158.17 ± 24.66 | 5.90 ± 13.52 | 377.93 ± 10.73 | 34.67 ± 9.44 |

### Example V

### Concentration of Multiple Microorganisms Spiked into Dechlorinated Tap Water

A new Fresenius F200NR hollow fiber filter was installed on the concentration system prior to each test. For each test, a sterile carboy was filled with 20 L of tap water and the tap water was dechlorinated with 80 g of sodium thiosulfate. A Masterflex I/P pump with I/P 73 tubing was connected to the inlet of the concentration system and used to pump the water from the carboy into the filter. A buffer reservoir containing the recovery fluid, 0.1 M PB+NaPP, was connected to the buffer line leading into the filter housing (outside of the filter fibers). A compressed air line was connected to a source of compressed air and then to a port on the concentration system leading to the center of the filter fibers. Flow rate was monitored using a flow meter integrated into the output tubing located post-filter (permeate side of filter). A PLC was used to control operation of the collection (filtration) and sample recovery steps.

For each test, microspheres and/or combinations of different microorganisms, including B. atrophaeus spores, Escherichia coli HB101-GFP and Enterococcus faecalis vegetative cells, MS2 bacteriophage and killed Cryptosporidium oocysts and Giardia cysts were spiked into a carboy containing 20 L of dechlorinated tap water. A minimum of three of these organisms was spiked into the carboy for each experiment. The inlet tubing leading into the concentration system was inserted into a peristaltic pump and the intake end was placed into the water in the carboy. The pump was set to pump at approximately 3.5 to 4 L/min. After the test fluid was pumped through the filter, 5 L of dechlorinated tap water was used to rinse the carboy and lines leading into the filter to ensure that as many spiked organisms as possible entered the filter. After filtration was complete, recovery fluid (PB+NaPP) was pumped through the filter in the forward flow direction. The amount of buffer used was sufficient to completely replace the test fluid left in the filter and filter housing after filtration. After a brief incubation, buffer was removed by pumping air through the filter in the forward flow direction. A sequence of buffer and air backflushes was then employed to remove material collected on the filter and deposit it in the collection vessel(s). The backflush sequence consisted of alternating both air backflushes through the center of the fibers only but in the reverse path of tap water entry to the filter fiber cores with PB+NaPP buffer backflushes from the outside of the fibers to the inside of the fibers. The retentate sample was collected in one or more collection vessels placed beneath the sample output port. Microspheres, Cryptosporidium oocysts and Giardia cysts were enumerated using epifluorescence microscopy. E. coli, and B. atrophaeus spores were enumerated by filtering aliquots of retentate through a 0.45 μm filter and plating on appropriate media. E. faecalis was enumerated using the IDEXX Enterolert system. MS2 bacteriophage were enumerated using the double agar overlay technique (Eaton, A. D., L. S. Clesceri, E. W. Rice, and A. E. Greenberg, 2010. Standard Methods for the Analysis of Water and Waste Water, 21st edition American Public Health Association, American Water Works Association, Water Environment Federation, Washington, D.C.). Data for runs involving simultaneous concentration of three or more organism are shown in Table 4.

TABLE 4
Data from experiments using the inventive method to recover
microspheres and multiple microorganisms from tap water.

| | | | Sample | | |
|---|---|---|---|---|---|
| | Quantity spiked | Volume | concentration | Retentate | |
| Test organism (n) | (CFU) | Filtered (L) | (CFU/ml) | Volume (ml) | % Recovery |
| Microspheres | 2.8 × 10⁶ | 10.37 ± 0.38 | 4819.68 ± 844.58 | 389.0 ± 11.4 | 66.39 ± 11.09 |
| (n = 5) | | | | | |
| Bacillus atrophaeus | 2716.8 ± 1420.2 | 25.0 ± 0 | 6.01 ± 5.33 | 395.83 ± 7.36 | 86.33 ± 56.64 |
| spores (n = 6) | | | | | |
| Enterococcus | 2677.5 ± 902.8 | 25.0 ± 0 | 5.05 ± 0.97 | 395.83 ± 7.36 | 78.27 ± 15.14 |
| faecalis (n = 6) | | | | | |
| Escherichia coli | 3422.2 ± 901.0 | 25.0 ± 0 | 3.36 ± 0.92 | 395.83 ± 7.36 | 44.95 ± 15.81 |
| HB101-GFP (n = 6) | | | | | |
| MS2 bacteriophage | 1.25 × 10⁵ ± 1.40 × 10⁵ | 25.0 ± 0 | 278.8 ± 420.0 | 395.0 ± 8.7 | 58.54 ± 42.9 |
| (n = 6) | | | | | |
| Cryptosporidium | 200 ± 2 | 25.0 ± 0 | 0.36 ± 0.23 | 395.0 ± 8.7 | 72.7 ± 47.3 |
| oocysts (n = 6) | | | | | |
| Giardia cysts (n = 6) | 200 ± 2 | 25.0 ± 0 | 0.26 ± 0.14 | 395.0 ± 8.7 | 51.8 ± 29.4 |

### Example VI

### Field Concentration of Indicator Organisms from River Water

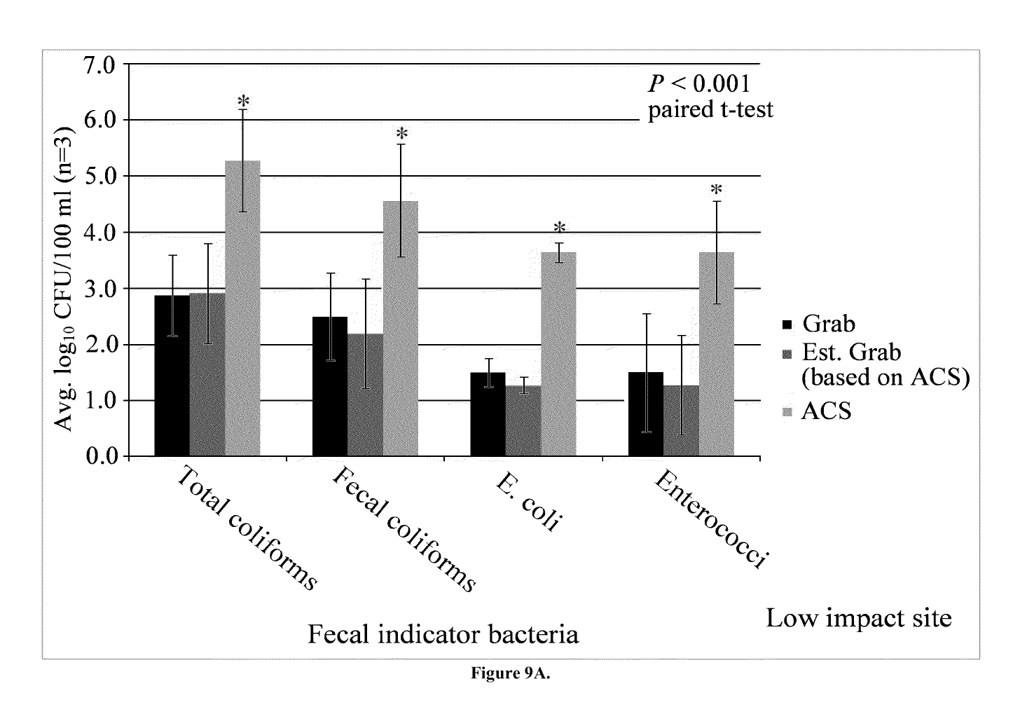

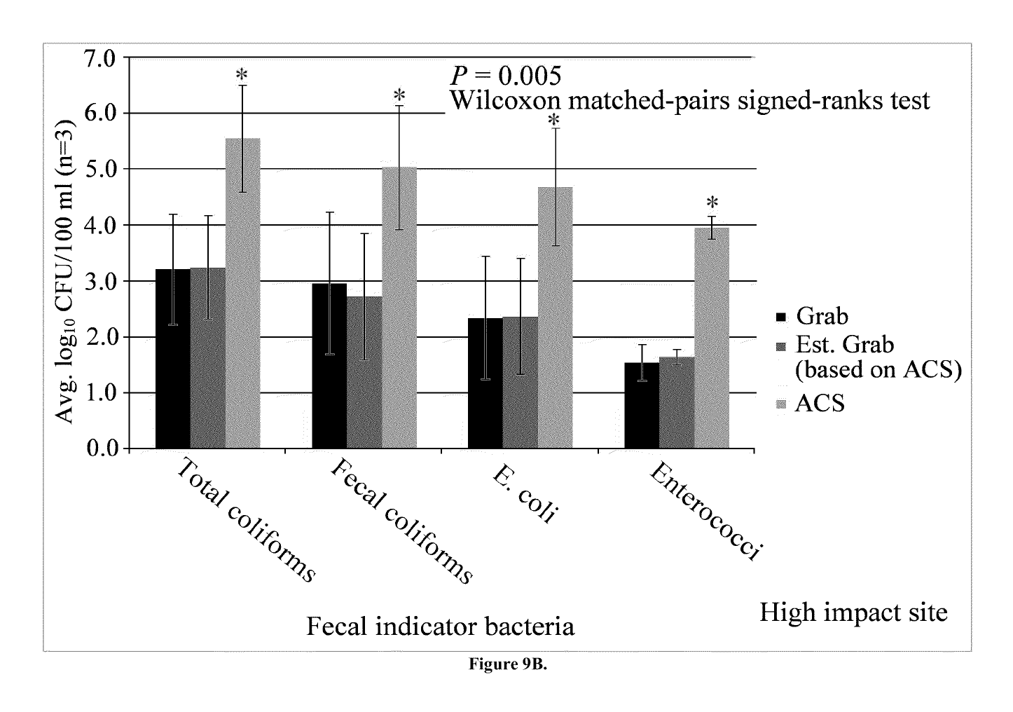

The concentration system was used to collect and concentrate organisms in 100 L of river water at two different locations, a low impact site with minimal expected contamination and a high impact site with a higher risk of contamination due to surrounding industrial and residential development. Retentate samples resulting from the concentration were screened for indicator organisms that are used to assess potential risk of contamination of water with pathogenic microorganisms. Results using the inventive system for sampling were compared to results obtained using a current standard method. For these tests, the concentration system was taken to the sites on the river for sample collection rather than bringing the 100 L water samples to the laboratory. This is considered advantageous because 1) 100 L samples are bulky and heavy, making them difficult to collect and transport and 2) the samples are closer to their natural state when processed on site versus the delay that is caused by the logistics of storing/transporting large carboys of water. Power for the field concentration was provided by two rechargeable emergency power sources. A length of flexible tubing was connected to the inlet of the concentration system, inserted through a Masterflex I/P peristaltic pump and dropped into the water. A weight was attached near the tubing intake to ensure that the tubing would stay below the water surface during collection. The pump was started, initiating uptake of the river water. After 100 L had been filtered, the concentration system was taken back to the laboratory for recovery of organisms collected within the filter. Sample recovery was done by injecting recovery fluid, (0.1 M PB+NaPP), into the filter using a port located just upstream of the filter inlet. Sufficient fluid was used to completely replace any tap water remaining in the filter and filter housing and recovery fluid was incubated with the filter for 10 minutes. A backflush sequence was initiated to remove collected particulates, including target indicator organisms, from the cores of the filter fibers. The backflush sequence consisted of alternating both air backflushes through the center of the fibers only but in the reverse path of tap water entry to the filter fiber cores with PB+NaPP buffer backflushes from the outside of the fibers to the inside of the fibers. The air and liquid backflush steps were alternated until about 400 ml of retentate sample was collected into one or more containers through a port located at the bottom of the filter that connected to the center of the fibers. A PLC was used to control all steps in the concentration procedure except turning on the peristaltic pump and the forward flow of buffer into the filter, which were done manually. The data in FIGS. 9A and B show a comparison of the inventive method and the standard method for three experiments at each of the two sampling sites. In these figures, ‘grab’ refers to samples collected using the standard method (without large volume filtration to collect a 1 L water sample), ‘ACS’ refers to water samples collected using the inventive system and ‘est. grab’ is a calculation to estimate what could be in the unfiltered river water based on the concentration in the ACS retentate sample and the retentate volume to filtered water volume ratio. This value assumes that 100% of the target organisms were recovered from the filter. As can be seen from the Figures, concentrations of all tested microbes were significantly higher after processing through the inventive system (ACS) for both low impact (minimal environmental contamination expected) and high impact (high levels of contamination expected) sites.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of an automated concentrator, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. An apparatus for extracting an analyte from a test fluid, comprising:
a concentration subsystem further comprising a filter having a first side and a second side, wherein the filter is selected to separate at least one analyte from the test fluid;
a test fluid input port disposed on the first side of the filter and in fluid communication with the test fluid source;
a test fluid output port disposed on the second side of the filter;
a plurality of backflush subsystems in fluid communication with the concentration subsystem, wherein the backflush subsystem further includes
a liquid backflush in fluid communication with the second side of the filter;
an air backflush comprising an air source in fluid communication with the first side of the filter;
said backflush subsystem adapted to remove at least one analyte from the filter thereby providing a sample, and
a sample output disposed on the first side of the filter.

2. The apparatus of claim 1, wherein the test fluid output port is in fluid communication with a filtrate output line.

3. The apparatus of claim 1, further comprising at least one pump disposed in fluid communication with at least the liquid backflush or air backflush, wherein the pumps are selected from the group consisting of a metering pump, a bellow, a double-diaphragm, a flexible impeller, a rotary lobe, a rotary vane, an oscillating, piston, a syringe, a nutating disc, a flexible liner, a progressing cavity, and a peristaltic pump.

4. The apparatus of claim 1, further comprising a plurality of valves disposed along at least one path of the test fluid, wherein the valves are selected from the group consisting of butterfly valves, trunnions, ball valves, plug valves, globe valves, needle valves, check valves, gate valves, angle seat piston valves, angle valves, ceramic disc valves, piston valves, pinch valves, or solenoid valves adapted to control the flow of the test fluid through the apparatus, and mass flow controllers.

5. The apparatus of claim 1, further comprising at least one pressure monitor disposed along at least one path of the test fluid, wherein the pressure monitor is selected from the group consisting of a transducer, piezo-resistive pressure sensor, piezo-resistive pressure transducer, miniature cylindrical pressure transducer, silicon strain gauge pressure transducer, pressure transmitter, digital pressure gauge, and analog pressure gauge.

6. The apparatus of claim 5, wherein the at least one transducer is disposed before the filter and after the filter.

7. The apparatus of claim 1, further comprising a sample detection system in fluid communication with the sample output port; wherein the sample detection system is selected from the group consisting of culture-dependent identification, biochemical identification, selective staining, immunoassay, shape-based identification, nucleic acid-based identification, sequence-based identification, carbohydrate-based identification, fatty acid-based identification, size-based identification, mass-selective identification, charge-selective identification, ion-selective identification, optical identification, spectral identification, ELISA, bioluminescence, electrochemiluminescence, growth selective media, growth differential media, fatty acid analysis, carbohydrate analysis, detection of toxin production, electrophoresis, fluorescent labeling with flow cytometry, optical differential staining, antibody capture with fluorescent antibody tagging; microarrays, nanoarrays, quartz crystal microbalance, nucleic acid sequence-based amplification, polymerase chain reaction, pyrosequencing, ion mobility spectroscopy, Raman spectroscopy, liquid chromatography-mass spectrometry, and combinations thereof.

8. The apparatus of claim 1, further comprising a large-particulate filter disposed along the path of the test fluid and before the test fluid reaches the filter.

9. The apparatus of claim 1, wherein the air source is selected from the group consisting of a pump in fluid communication with an air-intake valve, and a pressurized gas container.

10. The apparatus of claim 9, further comprising an air filter disposed on the air-intake valve.

11. The apparatus of claim 1, wherein the liquid backflush further comprises a pump in fluid communication with a liquid solution reservoir.

12. The apparatus of claim 1, further comprising a spiking port disposed along the path of the test fluid and before the test fluid reaches the filter.

13. The apparatus of claim 12, further comprising a mixing chamber disposed on the test fluid input line after the spiking port, wherein the mixing chamber is adapted to generate mild turbulence and approximate the natural dilution of water contamination.

14. The apparatus of claim 1, further comprising a cleaning subsystem, wherein the cleaning subsystem further comprises a pump and cleaning solution reservoir in fluid communication with the filter.

15. The apparatus of claim 13, further comprising a heating element adapted to heat a cleaning solution before the cleaning solution is introduced to the filter.

16. The apparatus of claim 1, further comprising a purge subsystem in fluid communication with the concentration subsystem, said purge subsystem adapted to release any gas in the filter.

17. The apparatus of claim 1, further comprising a forward flow buffer subsystem; wherein the forward flow buffer subsystem comprises a solution reservoir in fluid communication with a pump, and wherein the pump is in fluid communication with a test fluid input line and before the test fluid input port.

18. The apparatus of claim 1, further comprising a forward flow air subsystem; wherein the forward flow air subsystem comprises an air source in fluid communication with a test fluid input line and before the test fluid input port.

19. The apparatus of claim 17, wherein the air source is selected from the group consisting of a pump in fluid communication with an air-intake valve, and a pressurized gas container.

20. A method of extracting an analyte from a test fluid, comprising the steps of:
providing a test fluid source;
providing a filter with a first side and a second side;
collecting an analyte in the filter by passing the test fluid along a first path of travel through a filter whereby the analyte is captured in the first side of the filter;
equilibrating the filter with a recovery fluid by flowing the recovery fluid in the same direction as the test fluid;
removing the equilibrating recovery fluid from the filter by flowing air in the same direction as the test fluid;
forming a retentate by initiating at least one backflush sequence to remove the analyte from the filter, wherein the at least one backflush sequence comprises at least one air backflush followed by a plurality of liquid backflushes; and
collecting the backflush liquid as a sample.

21. The method of claim 20, wherein the gas is ambient air.

22. The method of claim 20, wherein the liquid is selected from the group consisting of water, a buffer and a solution.

23. The method of claim 20, further comprising the step of pumping a cleaning solution through the filter.

24. The method of claim 23, wherein the cleaning solution passes through the filter in the same path of travel as the fluid.

25. The method of claim 20, further comprising the step of purging any gas accumulated in the filter.

26. The method of claim 20, further comprising the step of delivering the sample to a sample detection system adapted to identify the analyte in the retentate; wherein the sample detection system is selected from the group consisting of culture-dependent identification, biochemical identification, selective staining, immunoassay, shape-based identification, nucleic acid-based identification sequence-based identification, carbohydrate-based identification, fatty acid-based identification, size-based identification, mass-selective identification, charge-selective identification, ion-selective identification, optical identification, spectral identification, ELISA, bioluminescence, electrochemiluminescence, growth selective media, growth differential media, fatty acid analysis, carbohydrate analysis, detection of toxin production, electrophoresis, fluorescent labeling with flow cytometry, optical differential staining, antibody capture with fluorescent antibody tagging; microarrays, nanoarrays, quartz crystal microbalance, nucleic acid sequence-based amplification, polymerase chain reaction, pyrosequencing, ion mobility spectroscopy, Raman spectroscopy, liquid chromatography-mass spectrometry, and combinations thereof.
